(19) United States
(12) Patent Application Publication
(10) Pub. No.: US 2010/0178670 A1
(43) Pub. Date: Jul. 15, 2010

(54) PROCESSES OF PRODUCING FERMENTATION PRODUCTS

(75) Inventors: Mads Torry Smith, Raleigh, NC (US); Frank Droescher Haagensen, Raleigh, NC (US)

Correspondence Address: NOVOZYMES NORTH AMERICA, INC., 500 FIFTH AVENUE, SUITE 1600, NEW YORK, NY, 10110, US

(73) Assignee: NOVOZYMES NORTH AMERICA, INC., Franklinton, NC (US)

(21) Appl. No.: 12/602,328

(22) PCT Filed: May 30, 2008
(86) PCT No.: PCT/US08/65270

§ 371 Date: Mar. 16, 2010

Related U.S. Application Data
(60) Provisional application No. 60/941,126, filed on May 31, 2007.

(51) Int. Cl. C12P 1/00

(52) U.S. Cl. 435/41

(57) ABSTRACT

The invention relates to a process of fermenting material derived from lignocellulose-containing material into a fermentation product by fermenting said material derived from lignocellulose-containing material using a fermenting organism obtained from a process of fermenting starch-containing material.

### TECHNICAL FIELD

The present invention relates to processes of producing a fermentation product from lignocellulose-containing material using a fermenting organism.

### BACKGROUND ART

Conversion of lignocellulose-containing material into fermentation products, such as ethanol, has the advantage of the ready availability of large amounts of feedstock, the desirability of avoiding burning or land filling the material, and the cleanliness of for instance ethanol fuel. Wood, agricultural residues, herbaceous crops, and municipal solid wastes have been considered as feedstock for, e.g., ethanol production. These materials primary consist of cellulose, hemicellulose, and lignin and are often referred to as “lignocellulose-containing materials” or “biomass,” which once converted to fermentable sugars easily can be fermented by a fermenting organism into a desired fermentation product.

Conventional processes of producing fermentation products from lignocellulose-containing materials typically include the following steps: pre-treatment, hydrolysis, fermentation, and optionally recovery of the fermentation product.

The structure of lignocellulose is not directly accessible to enzymatic hydrolysis. Therefore, the lignocellulose-containing material has to be pre-treated, e.g., by acid hydrolysis under adequate conditions of pressure and temperature, in order to break the lignin seal and disrupt the crystalline structure of cellulose. This causes solubilization of the hemicellulose and cellulose fractions. The cellulose and hemicelluloses can then be hydrolyzed enzymatically, e.g., by cellulolytic enzymes, to convert the carbohydrate polymers into fermentable sugars which may be fermented into desired fermentation products. Optionally the fermentation product may be recovered, e.g., by distillation.

Production of fermentation products, such as ethanol, from lignocellulose-containing material (“biomass”) is still too costly. Therefore, there is a need for providing processes that reduce the cost of producing a desired fermentation product from lignocellulose-containing material.

### SUMMARY OF THE INVENTION

The present invention relates to processes of producing a fermentation product from lignocellulose-containing material using a fermenting organism.

In the first aspect the invention relates to processes of fermenting materials derived from lignocellulose-containing materials into a fermentation product comprising fermenting said material using a fermenting organism obtained from a process of fermenting starch-containing materials, e.g., an ethanol production process.

The invention also relates to the use of a fermenting organism obtained from a process of fermenting starch-containing material in a process of fermenting material derived from lignocellulose-containing material into a fermentation product.

### BRIEF DESCRIPTION OF THE DRAWING

FIG. 1 shows weight loss for the PCS fermentations as a function of pitch level and time.

FIG. 2 shows a comparison of ethanol equivalents of glucose consumed after 24 hours of PCS fermentation compared to the realized ethanol equivalents.

FIG. 3 shows glycerol production during day 1 and day 2 of PCS fermentations.

FIG. 4 shows the glucose and ethanol conversion after 48 hours as a function of mash age and pitch.

### DETAILED DESCRIPTION OF THE INVENTION

In general fermenting organisms capable of producing a fermentation product from fermentable sugars, including glucose, are grown under precise conditions at a particular growth rate. When the fermenting organism is introduced into or added to the fermentation medium the inoculated fermenting organism passes through a number of stages. The initial period is referred to as the “lag phase” and may be considered a period of adaptation. During the next phase referred to as the “exponential phase” the growth rate gradually increases. After a period of maximum growth the rate ceases and the fermenting organism enters “stationary phase.” After a further period of time the fermenting organism enters the “death phase” where the number of viable cells declines.

### Fermentation Process of the Invention

The present invention relates to processes of fermenting materials derived from lignocellulose-containing materials, preferably pre-treated lignocellulose-containing materials, especially hydrolyzed pre-treated lignocellulose-containing materials, into a desired fermentation product.

More precisely the invention relates to processes of fermenting material derived from lignocellulose-containing material into a fermentation product comprising fermenting said material in the presence of a fermenting organism obtained from a process of fermenting starch-containing material. The fermenting organism may, for instance, be any of the ones disclosed in the “Fermenting Organism”-section below. In a specifically contemplated preferred embodiment the fermenting organism is yeast obtained from a process of fermenting starch-containing material into an alcohol, such as ethanol. The material to be fermented may preferably be pre-treated and/or hydrolyzed lignocellulose-containing material. In the case of, e.g., ethanol production, the material would contain fermentable sugars, such as glucose, fructose, maltose, xylose, mannose and/or arabinose, which can be fermented directly or indirectly into a desired fermentation product. One of the advantages of the invention is that the fermenting organism can be re-used. This reduces the cost for fermenting organism(s) and thus reduces the cost of producing a desired fermentation product, such as ethanol.

The fermenting organism may be transferred from the starch-based fermentation process to the process of fermenting lignocellulose-containing material in any suitable manner. In cases where there are productions based on both starch-containing material and lignocellulose-containing material on the same production site the fermenting organism(s) may simply be led from the starch-containing material based fermentation tank/vessel to the lignocellulose-containing material based fermentation tank/vessel. In one embodiment the fermenting organism from the starch-based fermentation may be added to the lignocellulose-based fermentation broth/medium in the form of fermentation organism-containing fermentation broth/medium. This means that the fermentation broth/medium is led directly from the starch-based fermentation tank/vessel to the lignocellulose-based fermentation tank/vessel.

According to the invention the starch and/or lignocellulose fermentation may be carried out in a tank/vessel of at least 50 liters. In embodiments of the invention the fermenting organism-containing fermentation broth/medium from the starch-based fermentation may constitute from 1-90 wt. %, preferably 2-80 wt. % such as 5-70 wt. %, such as preferably 1-25 wt. %, more preferably 2-20 wt. %, such as 5-10 wt. % of the lignocellulose-containing material to be fermented. The fermenting organism from the starch-based fermentation may in one preferred embodiment be used as the only source of fermenting organism. In another embodiment the fermenting organism from the starch-based fermentation may be a supplement to fermenting organism (culture) inoculated directly from a (e.g., conventional) propagation tank. The fermenting organism-containing fermentation broth/medium from the starch-based fermentation may constitute from above 0-100%, preferably 30-100 wt. %, preferably 50-100 wt. %, more preferably 80-100 wt. %, especially 90-100 wt. % of the total amount of fermenting organism inoculate used for fermenting the material (pre-treated and/or hydrolyzed material) derived from lignocellulose-containing (starting) material.

Alternatively, the fermenting organism may be separated, isolated and/or concentrated using any suitable means, e.g., by centrifugation and/or filtration, from the starch-based fermentation broth/medium before transferred and used for fermenting the lignocellulose derived material. For instance, separation, isolation and/or concentration may result in removal of a significant portion of the liquid part of the fermentation broth/medium. Any un-desired components in the starch-based fermentation broth/medium may be removed. The broth/medium may also be treated in any suitable way. In an embodiment centrifugation is carried out using a decanter centrifuge. Filtration may be done using a filter press, such as a plate or frame filter press. However, in a preferred embodiment the fermenting organism-containing fermentation broth/medium is used as is, i.e., without any treatment or, e.g., concentration at all.

The fermenting organism may be taken from the starch-based fermentation and added to the lignocellulose-based fermentation after the lag phase of the fermenting organism in the starch-based fermentation. In embodiments the fermenting organism is in any of the “exponential phase”, “stationary phase” and/or “death phase” when transferred/introduced into the lignocellulose-containing material based fermentation process of the invention. In a preferred embodiment the fermenting organism is in the exponential phase when transferred into the lignocellulose-containing material based fermentation process of the invention. In a preferred embodiment the fermenting organism is in the stationary phase when transferred into the lignocellulose-containing material based fermentation process of the invention.

In a specific embodiment the fermenting organism(s) is (are) taken from the starch-based fermentation and added to the lignocellulose-based fermentation after 2 to 72 hours of fermentation, such as after 2 hours, such as after 12 hours, such as after 24 hours or after 48 hours fermentation. For instance, Example 1 illustrates that if a fast fermentation is desired it is advantageous to use a 12 hour mash over other math propagates.

In an embodiment the fermenting organism is added to the lignocellulose-containing material based fermentation so that the viable yeast count per mL of fermentation broth/medium is in the range from 10⁵ to 10¹², preferably from 10⁷ to 10¹⁰, especially about 5×10⁷. It is to be understood that according to the invention the fermenting organism or fermenting organism-containing fermentation broth/medium may be added to the pre-treated and/or hydrolyzed lignocellulose-containing material before initiation of fermentation and/or during fermentation.

In a further embodiment the fermenting organism obtained from the starch-containing material based fermentation process and added to the (first) lignocellulose-containing material based fermentation process, in accordance with the first aspect of the invention, may be used to pitch further lignocellulose-containing material based fermentation processes. It is contemplated that the fermenting organism(s) added from the (first) lignocellulose-containing material based fermentation process may constitute from 1-90 wt. %, preferably 2-80 wt. % such as 5-70 wt. %, such as preferably 1-25 wt. %, more preferably 2-20 wt. %, such as 5-10 wt. % of the lignocellulose-containing material to be fermented in the further (especially second) lignocellulose-containing material based fermentation process. Also here the fermenting organism is preferably taken from after the lag phase, preferably from the exponential and/or stationary phases.

In an embodiment the invention relates to processes wherein at least a part of the fermenting organism used, as defined above, is obtained from a process of producing a fermentation product from starch-containing material, comprising the steps of:
i) liquefying starch-containing material,
ii) saccharifying the liquefied material with one or more carbohydrate-source generating enzymes,
iii) fermenting the saccharified material with a fermenting organism.

Steps ii) and iii) may be carried out simultaneously or sequentially. In step i) the starch-containing material is heated to a temperature above the gelatinization temperature. The gelatinized starch-containing material in step i) may be liquefied by subjecting the starch-containing material to an alpha-amylase. In an embodiment the starch-containing material may be liquefied using an alpha-amylase, preferably a bacterial alpha-amylase, especially a Bacillus alpha-amylase. The Bacillus alpha-amylase in preferred embodiments may be derived from a strain of Bacillus amyloliguefaciens, Bacillus licheniformis, Bacillus stearothermophilus or Bacillus subtilis. The liquefied material in step ii) may be saccharified using a carbohydrate-source generating enzyme, preferably a glucoamylase. The glucoamylase may be derived from a strain of Aspergillus, including Aspergillus awamori or Aspergillus niger, or Talaromyces, such as Talaromyces emersonii; or a strain of Athelia, such as Athelia rolfsii.

In an embodiment the invention relates to processes wherein at least a part of the fermenting organism is obtained from a process of producing a fermentation product from starch-containing material, comprising the steps of:
(a) saccharifying starch-containing material with one or more carbohydrate-source generating enzymes at a temperature below the initial gelatinization temperature of said starch-containing material,
(b) fermenting using a fermenting organism.

In an embodiment step (a) may further be carried out in the presence of an alpha-amylase, preferably a fungal alpha-amylase, especially an acid fungal alpha-amylase. The alpha-amylase in preferred embodiments may be derived from a strain of the genus Aspergillus, including Aspergillus kawachii, Aspergillus niger, and Aspergillus oryzae, or a strain derived from the genera Meripilus and Rhizomucor, preferably a strain of Meripilus giganteus or Rhizomucor pusillus (WO 2004/055178 incorporated by reference). The carbohydrate-source generating enzyme may preferably be a glucoamylase. The glucoamylase may be one or more of the ones mentioned above including Leucopaxillus giganteus, Pachylkytospora papyracea, and Trametes cingulate, all disclosed in WO 2008/069289.

### Starch-Containing Materials

Generally, the starch-containing material concerned may be any starch-containing material, including but not limited to cereals, preferably whole grain, from corn, cassava, wheat, barley, rye, milo, and potatoes; or any combination thereof.

### Lignocellulose-Containing Materials (Biomass)

Any suitable lignocellulose-containing material contemplated in context of the present invention. Lignocellulose-containing material may be any material containing lignocellulose. In a preferred embodiment the lignocellulose-containing material contains at least 50 wt. %, preferably at least 70 wt. %, more preferably at least 90 wt. % lignocellulose. It is to be understood that the lignocellulose-containing materials may also comprise other constituents such as cellulosic material, such as cellulose or hemicellulose, and can also comprise constituents such as sugars, such as fermentable sugars and/or un-fermentable sugars, proteins, etc. In context of the present invention the contemplated material will be referred to as “lignocellulose-containing material” or alternatively “biomass.”

Lignocellulose is a heterogeneous complex of carbohydrate polymers (cellulose and hemicellulose) and lignin. Lignin is an insoluble high molecular weight material of aromatic alcohols that strengthens lignocellulose. In general lignin contains three aromatic alcohols (coniferyl alcohol, sinapyl and p-coumaryl). In additions, grass and dicot lignin also contain large amounts of phenolic acids such as p-coumaric and ferulic acid, which are esterified to alcohol groups of each other and to other alcohols such as sinapyl and p-coumaryl alcohols. Lignin is further linked to both hemicelluloses and cellulose forming a physical seal around the latter two components that is an impenetrable barrier preventing penetration of solutions and enzymes (Howard et al., 2003, African Journal of Biotechnology 2 (12): 602-619).

Cellulose is a polymer of the simple sugar glucose covalently bonded by beta-1,4-linkages. Cellulose, like starch, is a homogenous polymer of glucose. However, unlike starch, the specific structure of cellulose favors the ordering of the polymer chains into tightly packed, highly crystalline structures, that are water insoluble and resistant to de-polymerization. Hemicellulose is, dependent on the species, a branched polymer of glucose or xylose, substituted with arabinose, xylose, galactose, furose, mannose, glucose or glucuronic acid (Mosier et al., 2005, Bioresource Technology 96: 673-686).

Lignocellulose-containing material is generally found, for example, in the stems, leaves, hulls, husks, and cobs of plants or leaves, branches, and wood of trees. Lignocellulose-containing material can also be, but is not limited to, herbaceous material, agricultural residues, forestry residues, municipal solid wastes, waste paper, and pulp and paper mill residues. It is understood herein that lignocellulose-containing material may be in the form of plant cell a material containing lignin, cellulose, and hemi-cellulose in a mixed matrix.

In an embodiment the lignocellulose-containing material is selected from the group of corn fiber, rice straw, pine wood, wood chips, poplar, wheat straw, switchgrass, bagasse, paper and pulp processing waste.

Other examples include corn stover, corn cobs, corn fiber, hardwood, such as poplar and birch, softwood, cereal straw, such as wheat straw, Miscanthus, municipal solid waste (MSW), industrial organic waste, office paper, or mixtures thereof.

In a preferred aspect, the material is corn stover and/or corn cobs. In another preferred aspect, the material is corn fiber.

### Pre-Treatment

The lignocellulose-containing material may advantageously be pre-treated before being hydrolyzed and/or fermented. In a preferred embodiment of the invention the pre-treated material is hydrolyzed, preferably enzymatically, before and/or during fermentation. Pre-treatment generally results in separation and/or release of cellulose, hemicellulose and/or lignin. The goal of pre-treatment is to improve the rate of enzymatic hydrolysis and/or increase the fermentation product yields. The lignocellulose-containing material to be fermented according to the invention may be subjected to pre-treatment using conventional methods well-known in the art. Pre-treatment may in a preferred embodiment take place in aqueous slurry. The material may during pre-treatment be present in an amount between 10-80 wt. %, preferably between 20-50 wt. %. A vast number of pre-treatment methods or combinations thereof are well-known in the art and may be used according to the invention.

Chemical Mechanical and/or Biological Pre-Treatment

The lignocellulose-containing material may be chemically, mechanically and/or biologically pre-treated before hydrolysis and/or fermentation. Mechanical treatment (often referred to as “physical” treatment) may be used alone or in combination with subsequent or simultaneous hydrolysis, especially enzymatic hydrolysis, to promote the separation and/or release of cellulose, hemicellulose and/or lignin.

Preferably, chemical, mechanical and/or biological pre-treatment is carried out prior to hydrolysis and/or fermentation. Alternatively, the chemical, mechanical and/or biological pre-treatment is carried out simultaneously with hydrolysis, such as simultaneously with addition of one or more cellulolytic enzymes, e.g., in combination with other enzyme activities mentioned below, to release fermentable sugars, such as glucose and/or maltose.

In an embodiment of the invention the pre-treated lignocellulose-containing material is detoxfied and/or washed. This may improve the fermentability of, e.g., dilute-acid hydrolyzed lignocellulose-containing material, such as corn stover and/or corn cobs. In one embodiment detoxification is carried out by steam stripping.

### Chemical Pre-Treatment

According to the present invention “chemical pre-treatment” refers to any chemical treatment which promotes the separation and/or release of cellulose, hemicellulose and/or lignin. Examples of suitable chemical pre-treatment steps include treatment with; for example, dilute acid, lime, alkaline, organic solvent, ammonia, sulphur dioxide, carbon dioxide. Further, wet oxidation and pH-controlled hydrothermolysis are also contemplated chemical pre-treatments.

Preferably, the chemical pre-treatment is acid treatment, more preferably, a continuous dilute and/or mild acid treatment, such as treatment with sulfuric acid, or another organic acid such as acetic acid, citric acid, tartaric acid, succinic acid, or mixtures thereof. Other acids may also be used. Mild acid treatment means in the context of the present invention that the treatment pH lies in the range from 1-5, preferably from pH 1-3. In a specific embodiment the acid concentration is in the range from 0.1 to 2.0 wt. % acid, preferably sulphuric acid. The acid may be mixed or contacted with the material to be fermented according to the invention and the mixture may be held at a temperature in the range of 160-220° C., such as 165-195° C., for periods ranging from minutes to seconds, e.g., 1-60 minutes, such as 2-30 minutes or 3-12 minutes. Addition of strong adds, such as sulphuric acid, may be applied to remove hemicellulose. This enhances the digestibility of cellulose.

Cellulose solvent treatment, also contemplated according to the invention, has been shown to convert about 90% of cellulose to glucose. It has also been shown that enzymatic hydrolysis could be greatly enhanced when the lignocellulosic structure is disrupted. Alkaline H₂O₂, ozone, organosolv (uses Lewis acids, FeCl₃, (Al)₂SO₄ in aqueous alcohols), glycerol, dioxane, phenol, or ethylene glycol are among solvents known to disrupt cellulose structure and promote hydrolysis (Mosier et al., 2005, Bioresource Technology 96: 673-686).

Alkaline chemical pre-treatment with base, e.g., NaOH, Na₂CO₃ and/or ammonia or the like, is also within the scope of the invention. Pre-treatment methods using ammonia are described in, e.g., WO 2006/110891, WO 2006/110899, WO 2006/110900, and WO 2006/110901, which are hereby incorporated by reference.

Wet oxidation techniques involve use of oxidizing agents, such as: sulphite based oxidizing agents or the like. Examples of solvent pre-treatments include treatment with DMSO (dimethyl sulfoxide) or the like. Chemical pre-treatment is generally carried out for 1 to 60 minutes, such as from 5 to 30 minutes, but may be carried out for shorter or longer periods of time dependent on the material to be pre-treated.

Other examples of suitable pre-treatment methods are described by Schell et al., 2003, Appl. Biochem and Biotechn. 105-108: 69-85, and Mosier et al., 2005, Bioresource Technology 96; 673-686, and U.S. Application Publication No. 2002/0164730, which references are hereby all incorporated by reference.

### Mechanical Pre-Treatment

As used in the present invention, the term “mechanical pre-treatment” refers to any mechanical or physical treatment which promotes the separation and/or release of cellulose, hemicellulose and/or lignin from lignocellulosic material. For example, mechanical pre-treatment includes various types of milling, irradiation, steaming/steam explosion, and hydrothermolysis.

Mechanical pre-treatment includes comminution (mechanical reduction of the particle size). Comminution includes dry milling, wet milling and vibratory ball milling. Mechanical pre-treatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is a batch-process, steam gun hydrolyzer system which uses high pressure and high temperature as defined above. A Sunds Hydrolyzer (available from Sunds Defibrator AB (Sweden) may be used for this.

### Combined Chemical and Mechanical Pre-Treatment

In a preferred embodiment both chemical and mechanical pre-treatment is carried out involving, for example, both dilute or mild acid treatment and high temperature and pressure treatment. The chemical and mechanical pre-treatment may be carried out sequentially or simultaneously, as desired.

Accordingly, in a preferred embodiment, the lignocellulose-containing material is subjected to both chemical and mechanical pre-treatment to promote the separation and/or release of cellulose, hemicellulose and/or lignin.

In a preferred embodiment the pre-treatment is carried out as a dilute and/or mild acid steam explosion step. In another preferred embodiment pre-treatment is carried out as an ammonia fiber explosion step (or AFEX pre-treatment step).

### Biological Pre-Treatment

As used in the present invention the term “biological pre-treatment” refers to any biological pre-treatment which promotes the separation and/or release of cellulose, hemicellulose, and/or lignin from the lignocellulosic material. Biological pre-treatment techniques can involve applying lignin-solubilizing microorganisms (see, for example, Hsu, 1996. Pretreatment of biomass, in Handbook on Bioethanol: Production and Utilization, Wyman, C. E., ed., Taylor & Francis. Washington, D.C., 179-212; Ghosh and Singh, 1993, Physicochemical and biological treatments for enzymatic/microbial conversion of lignocellulosic biomass, Adv. Appl. Microbiol. 39: 295-333: McMillan, 1994, Pretreating lignocellulosic biomass: a review, in Enzymatic Conversion of Biomass for Fuels Production, Himmel, Baker, and Overend, eds., ACS Symposium Series 566, American Chemical Society, Washington, D.C., chapter 15: Gong, Cao, Du, and Tsao, 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, 1996, Fermentation of lignocellulosic hydrolysates for ethanol production, Enz. Microb. Tech. 18: 312-331; and Vallander and Eriksson, 1990. Production of ethanol from lignocellulosic materials: State of the art, Adv. Biochem. Eng./Biotechnol. 42: 63-95).

### Hydrolysis

Before and/or during the fermentation process of the invention the lignocellulose-containing material, preferably pre-treated lignocellulose-containing material may be hydrolyzed in order to break the lignin seal and disrupt the crystalline structure of cellulose. In a preferred embodiment hydrolysis is carried out enzymatically. According to the invention the lignocellulose-containing material, preferably pre-treated lignocellulose-containing material, to be fermented has been or is hydrolyzed by one or more hydrolases (class EC 3 according to Enzyme Nomenclature), preferably one or more carbohydrases selected from the group consisting of cellulase, hemicellulase, amylase, protease, esterase, such as alpha-amylase, glucoamylase, proteases and lipases.

The enzyme(s) used for hydrolysis is capable of directly or indirectly converting carbohydrate polymers into fermentable sugars, such as glucose, fructose, maltose, xylose, mannose and/or arabinose, which can be fermented into a desired fermentation product, such as ethanol.

In an embodiment hydrolysis is carried out using cellulolytic enzymes. In a preferred embodiment hydrolysis is carried out using a cellulolytic enzyme preparation further comprising one or more polypeptides having cellulolytic enhancing activity. In a preferred embodiment the polypeptide(s) having cellulolytic enhancing activity is (are) of family GH61A origin. Examples of suitable and preferred cellulolytic enzyme preparations and polypeptides having cellulolytic enhancing activity are described in the “Cellulolytic Enzymes” section and “Cellulolytic Enhancing Polypeptides” sections below.

Hemicellulose polymers can be broken down by hemicellullolytic enzymes and/or acid hydrolysis to release its five and six carbon sugar components. The six carbon sugars (hexoses), such as glucose, galactose, arabinose, and mannose, can readily be fermented to fermentation products such as ethanol, acetone, butanol, glycerol, citric acid, fumaric acid etc. by suitable fermenting organisms including yeast.

The enzymatic treatment may be carried out in a suitable aqueous environment under conditions which can readily be determined by one skilled in the art. In a preferred embodiment hydrolysis is carried out at optimal conditions for the enzyme(s) in question.

Suitable process time, temperature and pH conditions etc. can readily be determined by one skilled in the art. Preferably, hydrolysis is carried out at a temperature between 30 and 70° C. preferably between 40 and 60° C., especially around 50° C. The process of the invention is preferably carried out at a pH in the range from 3-8, preferably pH 4-6, especially around pH 5. Preferably, hydrolysis is carried out for between 8 and 72 hours, preferably between 12 and 48 hours, especially around 24 hours.

Fermentation of lignocellulose derived material is carried out in accordance with a fermentation method of the invention as described above. In a preferred embodiment the carbohydrase has cellulolytic enzyme activity. Suitable enzymes are described in the “Enzymes” section below.

### Enzymes

Even if not specifically mentioned context of a process of the invention, it is to be understood that the enzyme(s) is (are) used in an effective amount.

### Cellulolytic Enzymes

The term “cellulolytic enzymes” as used herein is understood as including cellobiohydrolases (EC 3.2.1.91), e.g., cellobiohydrolase I and cellobiohydrolase II, as well as endo-glucanases (EC 3.2.1.4) and beta-glucosidases (EC 3.2.1.21). See relevant sections below with additional details on such enzymes.

In order to be efficient, the digestion of cellulose may require several types of enzymes acting cooperatively. At least three categories of enzymes are generally needed to convert cellulose into glucose: endoglucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is defined herein as a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

The cellulolytic enzyme may comprise a carbohydrate-binding module (CBM) which enhances the binding of the enzyme to a lignocellulose-containing fiber and increases the efficacy of the catalytic active part of the enzyme. A CBM is defined as contiguous amino acid sequence within a carbohydrate-active enzyme with a discreet fold having carbohydrate-binding activity. For further information of CBMs see the CAZy internet server (Supra) or Tomme et al., 1995, in Enzymatic Degradation of Insoluble Polysaccharides (Saddler and Penner, eds.), Cellulose-binding domains: classification and properties, pp. 142-163, American Chemical Society: Washington.

In a preferred embodiment the cellulolytic enzymes may be a cellulolytic preparation defined in U.S. application No. 60/941,251, which is hereby incorporated by reference. In a preferred embodiment the cellulolytic preparation comprising a polypeptide having cellulolytic enhancing activity (GH61A), is preferably Thermoascus aurantiacus GH61A disclosed in WO 2005/074656 (hereby incorporated by reference). The cellulolytic preparation may further comprise a beta-glucosidase, such as a beta-glucosidase derived from a strain of the genus Aspergillus, Penicillium, or Trichoderma, including the Humicola insolens CEL45A endoglucanase core/Aspergillus oryzae beta-glucosidase fusion protein disclosed in U.S. application Ser. No. 11/781,151 or PCT/US2007/074038 (Novozymes). In an embodiment the cellulolytic, preparation may also comprises a CBH II, preferably Thielavia terrestris cellobiohydrolase II (CEL6A). In an embodiment the cellulolytic preparation also comprises a cellulase enzymes preparation, preferably the one derived from Trichoderma reesei.

The cellulolytic activity may, in a preferred embodiment, be derived from a fungal source, such as a strain of the genus Trichoderma, preferably a strain of Trichoderma reesei; or a strain of the genus Humicola, such as a strain of Humicola insolens; or a strain of Chrysosporium, preferably a strain of Chrysosporium lucknowense.

In an embodiment the cellulolytic enzyme preparation comprises a polypeptide having cellulolytic enhancing activity (GH61A) disclosed in WO 2005/074656; a cellobiohydrolase, such as Thielavia terrestris cellobiohydrolase II (CEL6A), a beta-glucosidase (e.g., the fusion protein disclosed in U.S. application No. 60/832,511 and cellulolytic enzymes, e.g., derived from Trichoderma reesei.

In an embodiment the cellulolytic enzyme preparation comprises a polypeptide having cellulolytic enhancing activity (GH61A) disclosed in WO 2005/074656: a beta-glucosidase (e.g., the fusion protein disclosed in U.S. application No. 60/832,511) and cellulolytic enzymes, e.g., derived from Trichoderma reesei.

In an embodiment the cellulolytic enzyme is the commercially available product CELLUCLAST® 1.5 L or CELLUZYME™ available from Novozymes A/S, Denmark or ACCELERASE™ 1000 (from Genencor Inc. USA),

A cellulolytic enzyme may be added for hydrolyzing the pre-treated lignocellulose-containing material. The cellulolytic enzyme may be dosed in the range from 0.1-100 FPU per gram total solids (TS), preferably 0.5-50 FPU per gram TS, especially 1-20 FPU per gram TS. In another embodiment at least 1 mg cellulolytic enzyme per gram total solids (TS), preferably at least 3 mg cellulolytic enzyme per gram TS, such as between 5 and 10 mg cellulolytic enzyme(s) is (are) used for hydrolysis.

### Endoglucanase (EG)

Endoglucanases (EC No. 3.2.1.4) catalyse endo hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts. The authorized name is endo-1,4-beta-D-glucan 4-glucano hydrolase, but the abbreviated term endoglucanase is used in the present specification. Endoglucanase activity may be determined using carboxymethyl cellulose (CMC) hydrolysis according to the procedure of Ghose, 1987, Pure and Appl. Chem. 59: 257-268.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

### Cellobiohydrolase (CBH)

The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydrolases are mentioned above including CBH I and CBH II from Trichoderma reseei; Humicola insolens and CBH II from Thielavia terrestris cellobiohydrolase (CELL6A)

Cellobiohydrolase activity may be determined according to the procedures described by Lever et at., 1972, Anal. Biochem. 47: 273-279 and by van Tilbeurgh et al., 1982, FEBS Letters 149: 152-158: van Tilbeurgh and Claeyssens, 1985, FEBS Letters 187: 283-288. The Lever et al. method is suitable for assessing hydrolysis of cellulose in corn stover and the method of van Tilbeurgh et al. is suitable for determining the cellobiohydrolase activity on a fluorescent disaccharide derivative.

### Beta-Glucosidase

The term “beta-glucosidase” means a beta-D-glucoside glucchydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi at al., 2002, J. Basic Microbiol. 42: 55-66, except different conditions were employed as described herein. One unit of beta-glucosidase activity is defined as 1 micromole of p-nitrophenol produced per minute at 50° C. pH 5 from 4 mM p-nitrophenyl-beta-D-glucopyranoside as substrate in 100 mM sodium citrate, 0.01% TWEEN® 20.

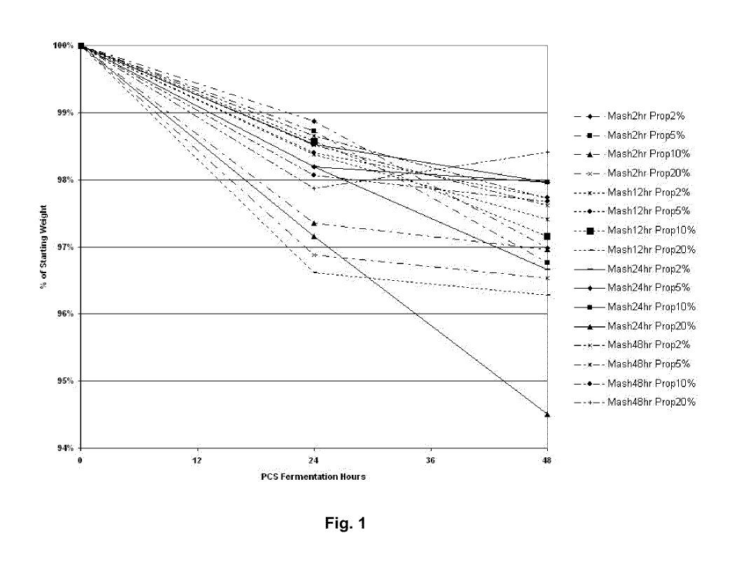

In a preferred embodiment the beta-glucosidase is of fungal origin, such as a strain of the genus Aspergillus, Penicillium or Trichoderma. In a preferred embodiment the beta-glucosidase is a derived from Trichoderma reesei, such as the beta-glucosidase encoded by the bgl1 gene (see FIG. 1 of EP 562003). In another preferred embodiment the beta-glucosidase is derived from Aspergillus oryzae (recombinantly produced in Aspergillus oryzae according to WO 02/095014). Aspergillus fumigatus (recombinantly produced in Aspergillus oryzae according to Example 22 of WO 02/095014) or Aspergillus niger (1981, J. Appl. 3: 157-163).

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose-containing material by cellulolytic protein under the following conditions: 1-50 mg of total protein/g of cellulose in PCS (pre-treated corn stover), wherein total protein is comprised of 80-99.5% w/w ceilulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more preferably at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Application Publication No. 2007/0077630 discloses an isolated polypeptide having cellulolytic enhancing activity and a polynucleotide thereof from Trichoderma reesei.

### Hemicellulolytic Enzymes

According to the invention the pre-treated lignocellulose-containing material may further be subjected to one or more hemicellulolytic enzymes, e.g., one or more hemicellulases.

Hemicellulose can be broken down by hemicellulases and/or acid hydrolysis to release its five and six carbon sugar components.

In an embodiment the lignocellulose derived material may be treated with one or more hemicellulases.

Any hemicellulase suitable for use in hydrolyzing hemicellulose, preferably into xylose, may be used. Preferred hemicellulases include xylanases, arabinofuranosidases, acetyl xylan esterase, feruloyl esterase, glucuronidases, endo-galactanase, mannases, endo or exo arabinases, exo-galactanses, and mixtures of two or more thereof. Preferably, the hemicellulase for use in the present invention is an exo-acting hemicellulases, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes A/S, Denmark).

In an embodiment the hemicellulase is a xylanase. In an embodiment the xylanase may preferably be of microbial origin, such as of fungal origin (e.g., Aspergillus, Fusarium, Humicola, Meripilus, Trichoderma) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes A/S, Denmark.

The hemicellulase may be added in an amount effective to hydrolyze hemicellulose, such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.06-0.10 g/kg DM substrate.

### Other Enzymes

Other hydrolytic enzymes may also be present during hydrolysis, fermentation, SSF, HHF or SHF. Contemplated enzymes include alpha-amylases; glucoamylases or another carbohydrate-source generating enzymes, such as beta-amylases, maltogenic amylases and/or alpha-glucosidases; proteases; or mixtures of two of more thereof.

### Fermentation of Lignocellulose Derived Material

Fermentation of lignocellulose-containing material may be carried out in any suitable way.

According to the invention fermentation may comprise pitching the pre-treated and/or hydrolyzed lignocellulose-containing material slurry with at least one fermenting organism capable of fermenting fermentable sugars, such as glucose and/or maltose.

Suitable conditions depend on the fermenting organism, the substrate and the desired product. One skilled in the art can easily determine what suitable fermentation conditions are.

### SSF, HHF and SHF

In a preferred embodiment hydrolysis and fermentation is carried out as a simultaneous hydrolysis and fermentation step (SSF). In general this means that combined/simultaneous hydrolysis and fermentation are carried out at conditions (e.g., temperature and/or pH) suitable, preferably optimal, for the fermenting organism(s) in question.

In another preferred embodiment hydrolysis step and fermentation step are carried out as hybrid hydrolysis and fermentation (HHF). HHF typically begins with a separate partial hydrolysis step and ends with a simultaneous hydrolysis and fermentation step. The separate partial hydrolysis step is an enzymatic cellulose saccharification step typically carried out at conditions (e.g., at higher temperatures) suitable, preferably optimal, for the hydrolyzing enzyme(s) in question. The subsequent simultaneous hydrolysis and fermentation step is typically carried out at conditions suitable for the fermenting organism(s) (often at lower temperatures than the separate hydrolysis step). Finally, the hydrolysis and fermentation steps may also be carded out a separate hydrolysis and fermentation, where the hydrolysis is taken to completion before initiation of fermentation. This is often referred to as “SHF”.

For yeast fermentations, such as with Saccharomyces cerevisae, the fermentation may be ongoing for 24 to 96 hours, in particular 35 to 60 hours. In an embodiment the fermentation is carded out at a temperature between 20 to 40° C., preferably 26 to 34° C., in particular around 32° C. In an embodiment the pH is from pH 3 to 6, preferably around pH 4 to 5.

The process of the invention may be performed as a batch or as a continuous process. The fermentation process of the invention may be conducted in an ultrafiltration system where the retentate is held under recirculation in presence of solids, water, and the fermenting organism and where the permeate is liquid containing the fermentation product. Equally contemplated is the process conducted in a continuous membrane reactor with ultrafiltration membranes and where the retentate is held under recirculation in presence of solids, water, the fermenting organism and where the permeate is a liquid containing the fermentation product.

After the fermentation the fermenting organism may be separated from the fermented slurry and recycled to the lignocellulose-containing slurry.

### Recovery

Subsequent to the fermentation the fermentation product may be separated from the fermented lignocellulose-containing slurry. The slurry may be distilled to extract the fermentation product or the fermentation product may be extracted from the fermentation medium/broth by micro or membrane filtration techniques. Alternatively the fermentation product may be recovered by stripping. Methods for recovery are well known in the art.

### Fermentation Products

The fermentation process of the invention may be used for producing any fermentation product, including alcohols (e.g., ethanol, methanol, and butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B12, beta-carotene); and hormones.

Also contemplated products include consumable alcohol industry products, e.g., beer and wine; dairy industry products, e.g., fermented dairy products; leather industry products and tobacco industry products.

In a preferred embodiment the fermentation product is an alcohol, especially ethanol and butanol. The fermentation product, such as ethanol, obtained according to the invention, may preferably be used as fuel. However, in the case of ethanol it may also be used as potable ethanol.

### Fermenting Organisms

The term “fermenting organism” refers to any organism, including bacteria and fungal organisms, such as yeast and filamentous fungi, suitable for producing a desired fermentation product. Especially suitable fermenting organisms according to the invention are able to ferment, i.e., convert sugars, such as glucose, fructose, maltose, xylose, mannose and/or arabinose, directly or indirectly into the desired fermentation product. Examples of fermenting organisms include fungal organisms, such as yeast. Preferred yeast includes strains of the genus Saccharomyces, in particular a strain of Saccharomyces cerevisiae or Saccharomyces uvarum; a strain of Pichia, in particular Pichia pastoris or Pichia stipitis; a strain of the genus Candida, in particular a strain of Candida arabinofermentans, Candida boidinii, Candida diddensii, Candida shehatae, Candida sonorensis, Candida tropicalis, or Candida utilis. Other contemplated yeast includes strains of Hansenula, in particular Hansenula anomala or Hansenula polymorpha; strains of Kluyveromyces, in particular Kluyveromyces fagilis or Kluyveromyces maixianus, and strains of Schizosaccharomyces, in particular Schizosaccharomyces pombe.

Preferred bacterial fermenting organisms include strains of Escherichia, in particular Escherichia coil, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenas, and strains of Thermoanaerobacter, in particular Thermoanaerobacter BG1L1 (Appl. Microbiol. Biotech. 77: 61-86) and Thermoanarobacter ethanolicus, Thermoanaerobacter mathranii, or Thermoanaerobacter thermosaccharolyticum. Strains of Lactobacillus are also envisioned as are strains of Bacillus thermoglucosidaisus, Corynebacterium glutamicum R, and Geobacillus thermoglucosidasius.

In an embodiment the fermenting organism is a C6 sugar fermenting organism, such as a strain of, e.g., Saccharomyces cerevisiae.

In connection with fermentation of lignocellulose derived materials, C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp. that are capable of fermenting C5 sugars include the ones concerned in, e.g., Ho et al., 1998, Applied and Environmental Microbiology, p. 1852-1859 and Karhumaa et al., 2006, Microbial Cell Factories 5:18.

In one embodiment the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

Commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Use

In an aspect the invention relates to the use of fermenting organism obtained from a process of fermenting starch-containing material in a process of fermenting material derived from lignocellulose-containing material into a fermentation product. The fermentation is done in accordance with the process of the invention. Contemplated lignocellulose-containing material, fermenting organisms, fermentation products etc. are described above.

### Materials & Methods

### Materials

Cellulolytic Preparation A: Cellulolytic composition comprising a polypeptide having cellulolytic enhancing activity (GH61A) derived from Thermoascus aurantiacus disclosed in WO 2005/074656; a beta-glucosidase (Humicola insolens CEL45A endoglucanase core/Aspergillus oryzae beta-glucosidase fusion protein disclosed in U.S. application Ser. No. 11/781,151; and cellulolytic enzymes preparation derived from Trichoderma reesei, Cellulolytic Preparation A is disclosed in co-pending U.S. application No. 60/941,251 (Novozyme).
PCS: NREL PCS (biomass number 43: acid-catalyzed, steam exploded)
Yeast: RED STAR™ available from Red Star/Lesaffre, USA

### Examples

### Example 1

Pre-Treated Corn Stover (PCS) Fermentation Inoculated with Corn Mash Propagate

This experiment was carried out to determine the effect on ethanol productivity of PCS fermentations inoculated with corn mash.

Unwashed acid-catalyzed, steam exploded PCS was hydrolyzed with cellulase (Cellulolytic Preparation A) at 1 L scale at 25 wt. % total solids (TB) in two separate reactors at 50° C. for 120 hours. The reactors were combined, giving a glucose concentration of approximately 89 g/L, and then filtered through a 0.45 micron Whatman fitter. Filtered hydrolyzate was utilized according to the experimental design in Table 1.

An overnight YPD prop (200 mg RED STAR™ yeast in 100 mL of media, 14 hours stirring in 32° C. environmental chamber) was prepared and used to inoculate a 500 g corn mash fermentation to a level of 5% w/w. Portions of the corn mash were removed at 2, 12, 24 and 48 hours. Broth was then used to inoculate 10 mL PCS hydrolysate fermentations at levels of 2, 5, 10 and 20% w/v.

Fermenters were saccharified for 0, 24 and 48 hours for each treatment condition and tested for sugars and ethanol on HPLC. Each condition was duplicated. Fermentations were monitored for weight toss. HPLC data was later corrected for weight loss. 24 hours mash at 10% was considered the control treatment.

TABLE 1
Experimental Design

| | Corn Mash | | % Corn | % PCS |
|---|---|---|---|---|
| Treatment | Fermentation Age hr | Replicate | Mash | Hydrolysate |
| 1 | 2 | 1 | 2 | 98 |
| 2 | 2 | 1 | 5 | 95 |
| 3 | 2 | 1 | 10 | 90 |
| 4 | 2 | 1 | 20 | 80 |
| 5 | 12 | 1 | 2 | 98 |
| 6 | 12 | 1 | 5 | 95 |
| 7 | 12 | 1 | 10 | 90 |
| 8 | 12 | 1 | 20 | 80 |
| 9 | 24 | 1 | 2 | 98 |
| 10 | 24 | 1 | 5 | 95 |
| 11 | 24 | 1 | 10 | 90 |
| 12 | 24 | 1 | 20 | 80 |
| 13 | 48 | 1 | 2 | 98 |
| 14 | 48 | 1 | 5 | 95 |
| 15 | 48 | 1 | 10 | 90 |
| 16 | 48 | 1 | 20 | 80 |
| 17 | 2 | 2 | 2 | 98 |
| 18 | 2 | 2 | 5 | 95 |
| 19 | 2 | 2 | 10 | 90 |
| 20 | 2 | 2 | 20 | 80 |
| 21 | 12 | 2 | 2 | 98 |
| 22 | 12 | 2 | 5 | 95 |
| 23 | 12 | 2 | 10 | 90 |
| 24 | 12 | 2 | 20 | 80 |
| 25 | 24 | 2 | 2 | 98 |
| 26 | 24 | 2 | 5 | 95 |
| 27 | 24 | 2 | 10 | 90 |
| 28 | 24 | 2 | 20 | 80 |
| 29 | 48 | 2 | 2 | 98 |
| 30 | 48 | 2 | 5 | 95 |
| 31 | 48 | 2 | 10 | 90 |
| 32 | 48 | 2 | 20 | 80 |

FIG. 1 shows the weight loss for the PCS fermentations as a function of pitch level and time. The higher pitch levels tend to have the greatest weight loss.

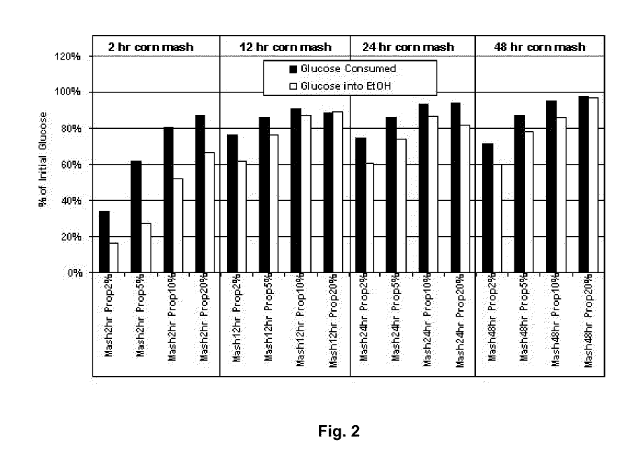

FIG. 2 shows the relative levels of glucose consumed compared to the amount of ethanol actually produced after 24 hours of fermentation. The PCS fermentations inoculated with 2 hours mash produced less ethanol than the other treatments. There is no significant difference between the 12, 24 and 48 hours corn mashes. Around 85% of the ethanol potential is reached with pitches of 10% w/v or greater for the three older corn mash propagates.

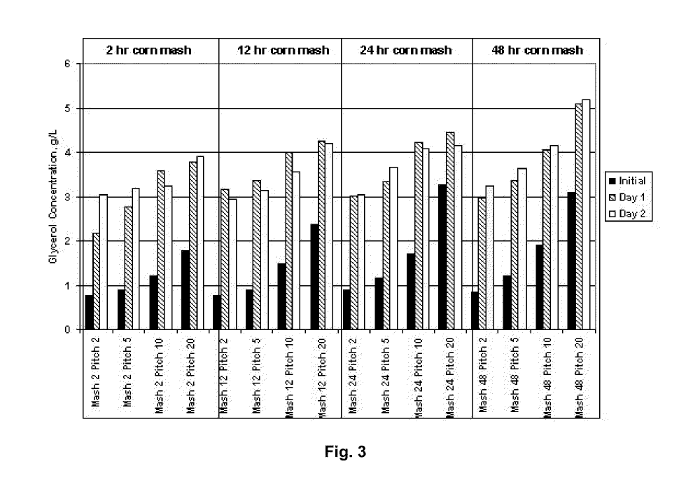

FIG. 3 illustrates glycerol production during the fermentations. The levels for the 2 hours mashes are on the same level as for other treatments, indicating that a significant amount of glucose went to biomass growth using 2 hours corn mash relative to the other propagates.

All fermentations produced measurable glycerol in the first 24 hours period and relatively little during the second 24 hours period.

The 12, 24 and 48 hours mash treatments exhibited similar glucose conversion efficiencies as the pitch level was increased. There is no clear difference between the 24 hours and 48 hours mash samples.

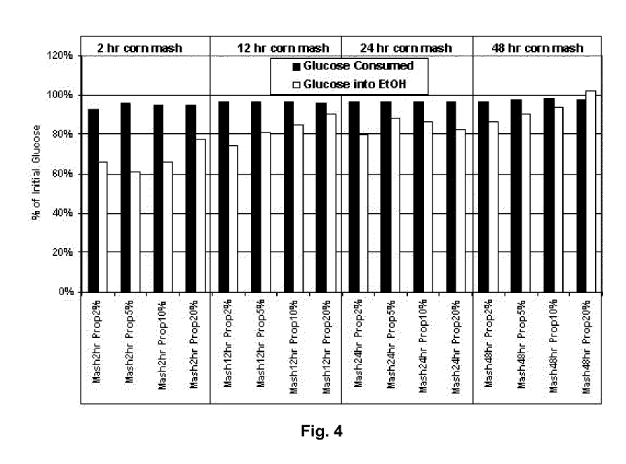

FIG. 4 shows the accumulated glucose and ethanol conversions after 48 hours of fermentation. More than 93% of the initial glucose was consumed within 48 hours for all corn mashes. The corn mash propagates produce most ethanol at higher pitch and the more mature the prop the higher the yield.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties.

1. A process of fermenting material derived from lignocellulose-containing material into a fermentation product comprising fermenting said material derived from lignocellulose-containing material using a fermenting organism obtained from a process of fermenting starch-containing material.

2. The process of claim 1, wherein a fermenting organism obtained from a process of fermenting starch-containing material is added to the material derived from lignocellulose-containing material.

3. The process of claim 1, wherein the lignocellulose-containing material is hydrolyzed before and/or during fermentation.

4. The process of claim 1, wherein the lignocellulose-containing material has been pre-treated or has been pre-treated or hydrolyzed enzymatically before fermentation.

5. The process of claim 1, wherein the fermenting organism is obtained from a process of fermenting starch-containing material into a fermentation product.

6. The process of claim 1, wherein the fermenting organism added to the material derived from lignocellulose-containing material is comprised in the fermentation broth from a process of fermenting starch-containing material into a fermentation product.

7. The process of claim 6, wherein the fermenting organism-containing fermentation broth constitutes from 1-90 wt. % of the lignocellulose-containing material to be fermented.

8. The process of claim 6, wherein the fermenting organism-containing fermentation broth constitutes from 50-100 wt. % of the total amount of fermenting organism inoculate used for fermentation.

9. The process of claim 1, wherein the fermenting organism is separated from the fermenting organism-containing fermentation broth before used for fermenting the material derived from lignocellulose-containing material.

10. The process of claim 1, wherein the fermenting organism-containing fermentation broth is concentrated before fermentation.

11. The process of claim 1, wherein the fermenting organism-containing fermentation broth is used as is.

12. The process of claim 1, wherein the fermenting organism or fermenting organism-containing fermentation broth is added to the material to be fermented before and/or during fermentation.

13. The process of claim 1, wherein the fermenting organism is taken from the process of fermenting starch-containing material and added to the material to be fermented after the fermenting organism has been in the lag phase.

14. The process of claim 1, wherein the lignocellulose-containing material has been chemically or mechanically and/or microbially pre-treated.

15. The process of claim 1, wherein the fermenting organism obtained from the starch-containing material based fermentation process and added to the first lignocellulose-containing material based fermentation process is added to a further lignocellulose-containing material based fermentation processes.

16. The process of claims 1, wherein the fermentation product is an alcohol.

17-19. (canceled)
